(19) United States
(12) Patent Application Publication
(10) Pub. No.: US 2007/0173816 A1
(43) Pub. Date: Jul. 26, 2007

(54) Device for aligning a human or animal vertebral column

(76) Inventor: Peter Metz-Stavenhagen, Bad Wildungen (DE)

Correspondence Address: WELSH & KATZ, LTD, 120 S RIVERSIDE PLAZA, 22ND FLOOR, CHICAGO, IL, 60606, US

(21) Appl. No.: 10/557,172

(22) PCT Filed: May 14, 2004
(86) PCT No.: PCT/DE04/01005

§ 371 Date: Oct. 2, 2006

(30) Foreign Application Priority Data
May 19, 2003 (DE) 203-07-773.3

(51) Int. Cl. A61F 2/30

(52) U.S. Cl. 606/61

(57) ABSTRACT

The invention relates to a device for aligning a human or animal vertebral column. Said device comprises a fixing bar (12, 40, 42), which fixes at least two vertebral bodies (44), can be attached to at least two fastening elements (16) that can be anchored to the vertebral column and can be secured to the fastening elements (16) by screws (20) or other fastening means. Longitudinal cavities (29, 29′) are configured in the fixing bar (12, 40, 42) in an axial direction. The screw (20) or other fastening means pass/passes through said cavities in order to fasten the fixing bar (12, 40, 42) to the fastening element (16).

### BACKGROUND OF THE INVENTION

1. Field of the Invention.

The present invention relates to a device for aligning a human or animal spine, said device having a fixation rod for fixing at least two vertebral bodies, said fixation rod being attachable to at least two fastening elements adapted to be anchored on the spine and being fastenable to the fastening elements by means of screws or other fastening means.

2. Description of the Prior Art.

Such a device, in which fastening elements for attachment of one, two or three fixation rods for aligning the spine are attached to the vertebral body, is known from DE 202 07 847.2. These fixation rods are implemented as threaded rods and may be utilized either as compression rods or as distraction rods. For example, these threaded rods are locked on the fastening element in a corresponding fixation rod mount, with said fixation rod mount being compulsorily dimensioned to be large to receive the fixation rod so that the fastening element is also of a certain size.

### BRIEF SUMMARY OF THE INVENTION

In view thereof it is the object of the present invention to provide a device of the type mentioned herein above that is easier to attach to the spine and that may be of smaller dimensions.

As a technical solution to this object, the invention suggests to further develop the device mentioned herein above so as to form in the fixation rod, in the axial direction thereof, long holes through which the screw, or the other fastening means for fastening the fixation rod to the fastening element, extends.

A device configured according to this technical solution has the advantage that thanks to this long hole the fixation rod can be readily fastened to the fastening element by means of a screw extending through said long hole. This eliminates the need for a complicated construction of the mount surrounding the fixation rod so that the entire device can be dimensioned much smaller.

Another advantage is that, by threading the screw through the long hole, the fixation rod is virtually retained from within and that the screw is no longer projecting beyond the fixation rod so that this also permits to achieve smaller dimensions.

Still another advantage is that, for implanting this device, the fixation rod may first be temporarily attached to the spine by slightly tightening the screw and that the spine may then be aligned quietly, prior to definitively tightening the screw and thus definitively fixing the fixation rod. This facilitates the alignment of the spine in the desired position.

In a preferred embodiment, the fastening element is configured to be planar on the side turned toward the fixation rod so that a contact surface is defined there. Thanks to this planar configuration, the fastening element can be configured to be a flat disc which contributes to achieving a minimum size. Another advantage is that such a fastening element can be manufactured at much lower cost because of its much simpler construction.

In another preferred embodiment, the distance between neighboring long holes is smaller than the length of the shortest of said long holes. This applies both to the case in which the two long holes have different sizes and to the case in which the two long holes are identically formed. This provision offers a plurality of possibilities for placing the screw over the entire length of the fixation rod so that it is made certain that the fixation rod can be attached to the fastening element in almost any position.

In a particularly preferred embodiment, the edge of the long hole is chamfered or conical so that, when countersunk screws are used for example, the screw head may be sunk at least partially into the long hole so that no or but small parts of the screw protrude from the fixation rod, which also contributes to further decrease the overall size of the device.

Further advantages of the device of the invention will become apparent in the appended drawings and in the following description of embodiments thereof. Likewise, the invention lies in each and every novel feature or combination of features mentioned above or described herein after. The embodiments discussed herein are merely exemplary in nature and are not intended to limit the scope of the invention in any manner.

### BRIEF DESCRIPTION OF THE SEVERAL VIEWS OF THE DRAWING

In the drawing:

FIG. 1 is a front exploded view of a device of the invention;

FIG. 2 is a side view of the device of FIG. 1;

FIG. 3 is a bottom view of a fastening element of FIG. 1;

FIG. 4a is a top view of a detail of the fixation rod of FIG. 1;

FIG. 4b is a top view of a second embodiment of the fixation rod;

FIG. 5 is a top view of the fastening element of FIG. 3;

FIG. 6 is a side view of a portion of a human spine with a first embodiment of a device of the invention;

FIG. 7 is a side view of a portion of a human spine with a second embodiment of a device of the invention;

FIG. 8 is a side view of a portion of a human spine with a third embodiment of a device of the invention.

### DETAILED DESCRIPTION OF THE INVENTION

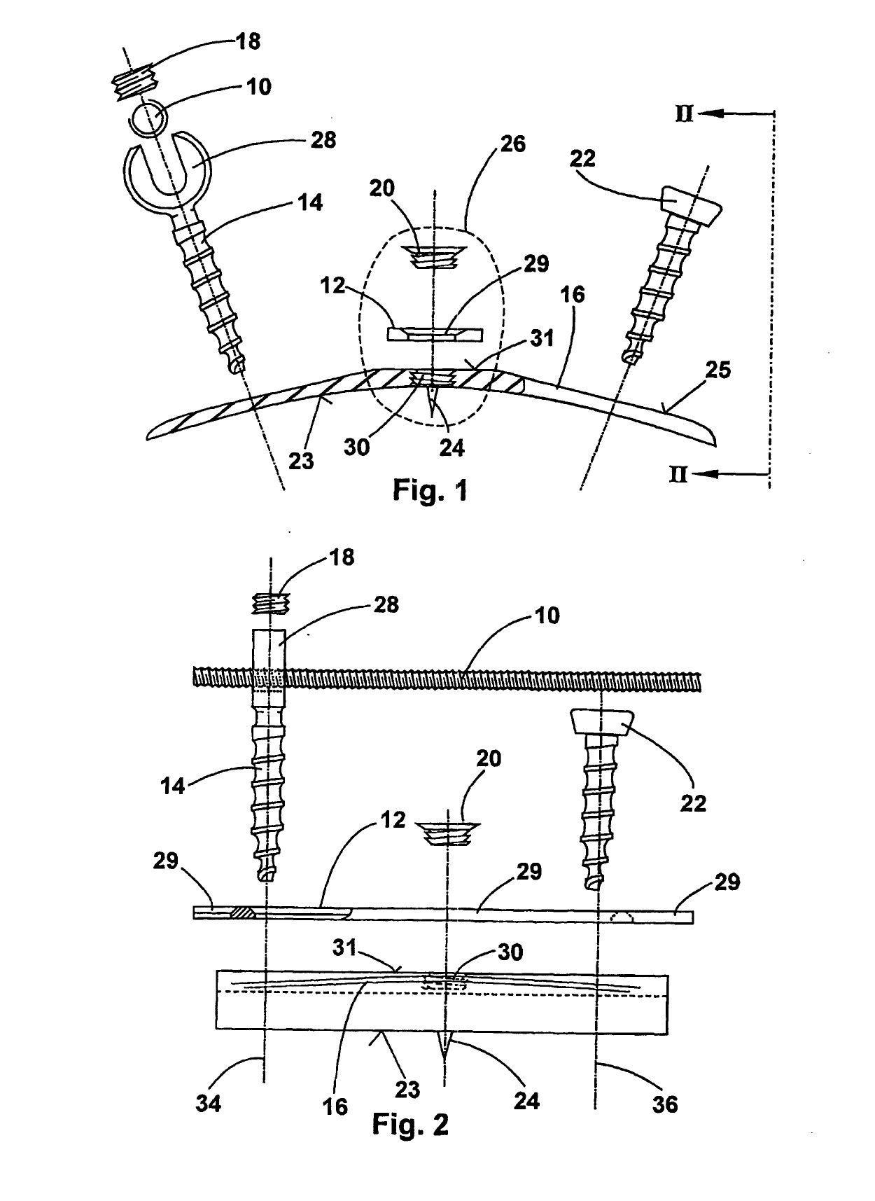

The FIGS. 1 and 2 are front and side exploded views of an embodiment of a device for fixing a human or animal spine in accordance with the invention, said device including a threaded rod 10, a flat fixation rod 12, a pedicle screw 14, a fastening element 16, a grub screw 18, a screw 20 and a cancellous screw 22. The fastening element 16 comprises on its underside 23 turned toward a vertebral body a pointed spike 24 and on its upper side 25 turned away from the vertebral body a fixation rod mount 26. The head of the pedicle screw 14 is configured to be the threaded rod mount 28.

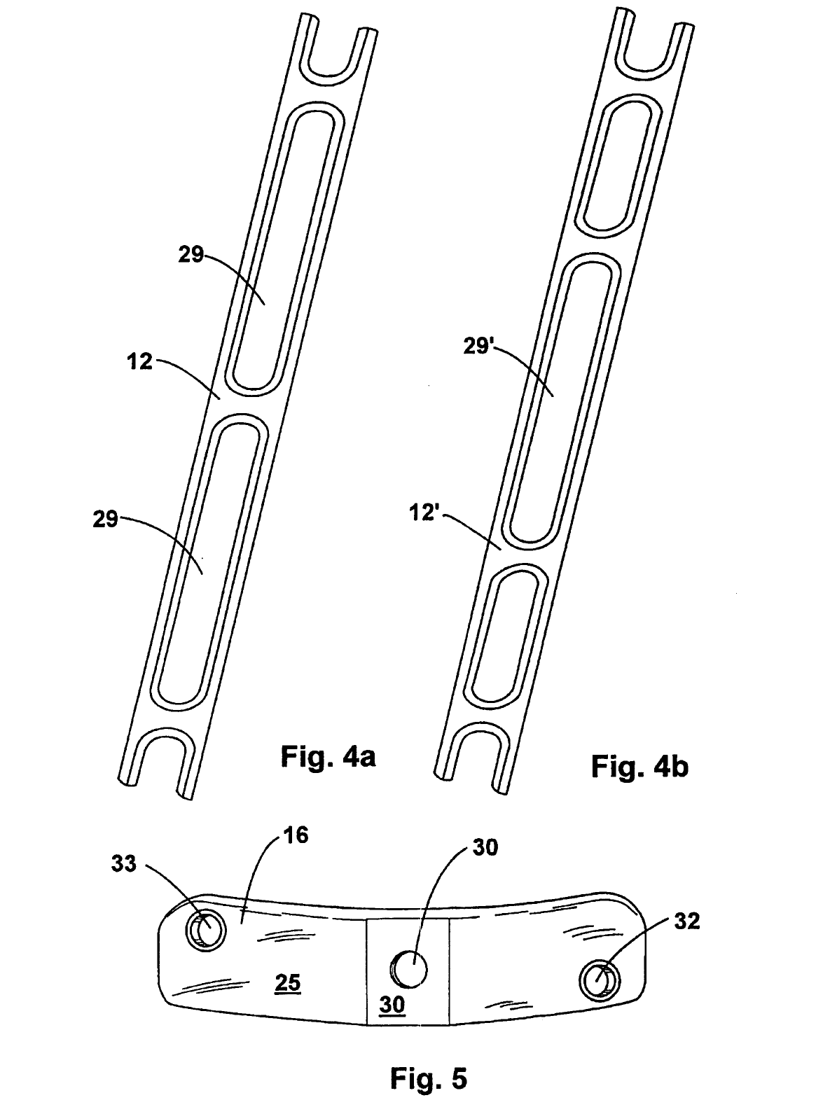

The fixation rod 12 is made from a flat tape and comprises a number of long holes 29 and 29′ as can be seen in detail in the FIGS. 4a and 4b. The width of the long holes 29, 29′ is chosen to allow close-fitting passage of the screw 20 therethrough. The length of the long holes 29, 29′ may vary depending on the application. In any case however, the long holes 29, 29′ are longer than the spacing between neighboring long holes 29, 29′. Neighboring long holes 29, 29′ may thereby have the same length as illustrated in FIG. 4a or different lengths as illustrated in FIG, 4b.

The fixation rod mount 26 is disposed on the fastening element 16 in the center thereof and extends from one longitudinal rim to the other substantially in the direction of the spine whilst the fastening element 16 is oriented substantially transversely to the spine. The fixation rod mount 26 includes the screw 20, a screw receiving chamber 30 and an abutment surface 31 for receiving the fixation rod 12 provided on the side turned away from the spine. Approximately in the center of the fixation rod mount 26 there is provided the threaded screw receiving chamber 30 for receiving the screw 20 by means of which the fixation rod 12 is fastened to the fastening element 16 in the desired position, with the fixation rod 12 thereby lying directly on the abutment surface 31 in order to reduce the overall height of the fastening element. The screw 20 comprises a buttress thread. The term buttress thread is meant to include not only the buttress metric thread DIN 513 but also buttress threads having a slightly larger or slightly smaller flank angle, a zero flank angle or a negative flank angle as well as buttress threads in accordance with EP 885 598.

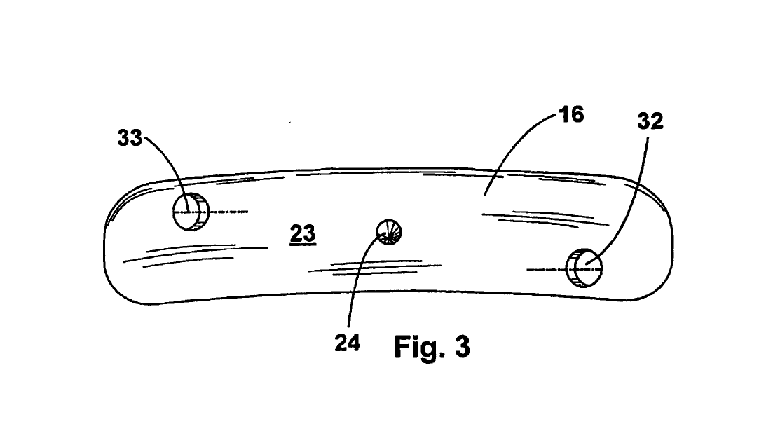

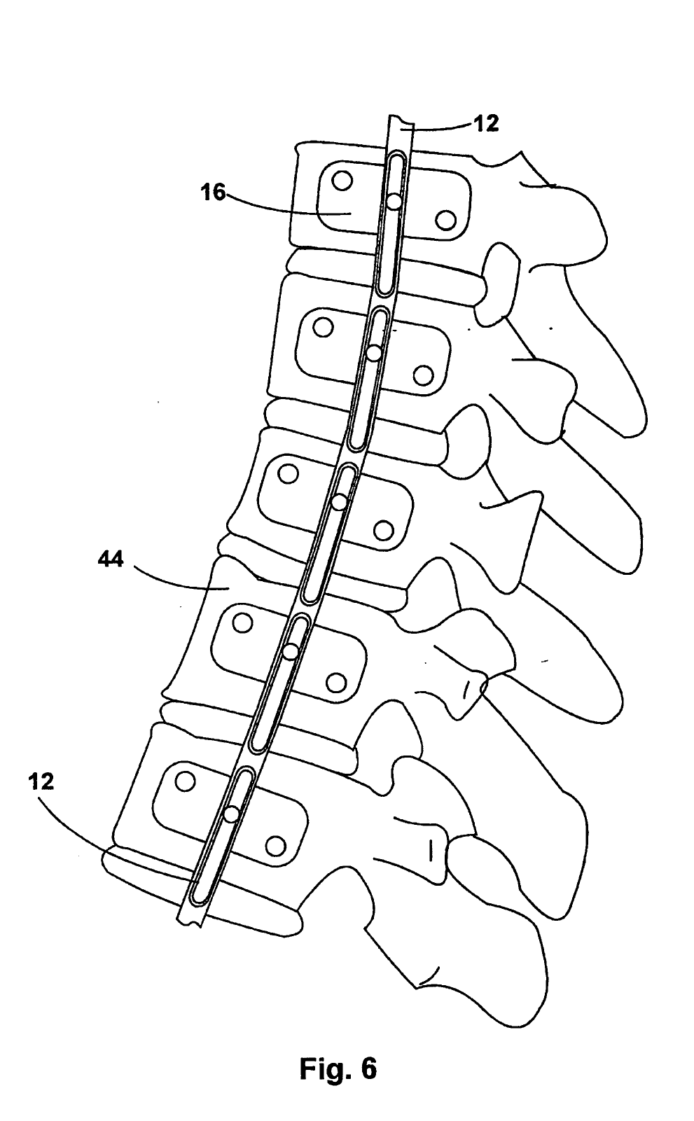

As can be seen in the FIGS. 3 and 5, there are provided, on the respective outer ends of the fastening element 16, two diagonally opposite screw receiving chambers 32, 33 that are dimensioned such that the shank of the pedicle screw 14 and of the cancellous screw 22 passes therethrough and that the respective screw head is fittingly received therein.

In another embodiment that has not been illustrated herein, there are provided in the fastening element more than two screw receiving chambers in order to provide for more screw points. In still another embodiment that has not been illustrated herein the fastening element is configured to have recesses or is even configured in the shape of a grid in order to achieve savings in weight and/or production cost.

According to the size and shape of the vertebral body, the fastening element 16 is configured to be arcuate in order to make it flush with the vertebral body. In the case of the fastening element 16 illustrated here, which is about 1.5 mm thick, the arch is about 6 mm high at its highest point.

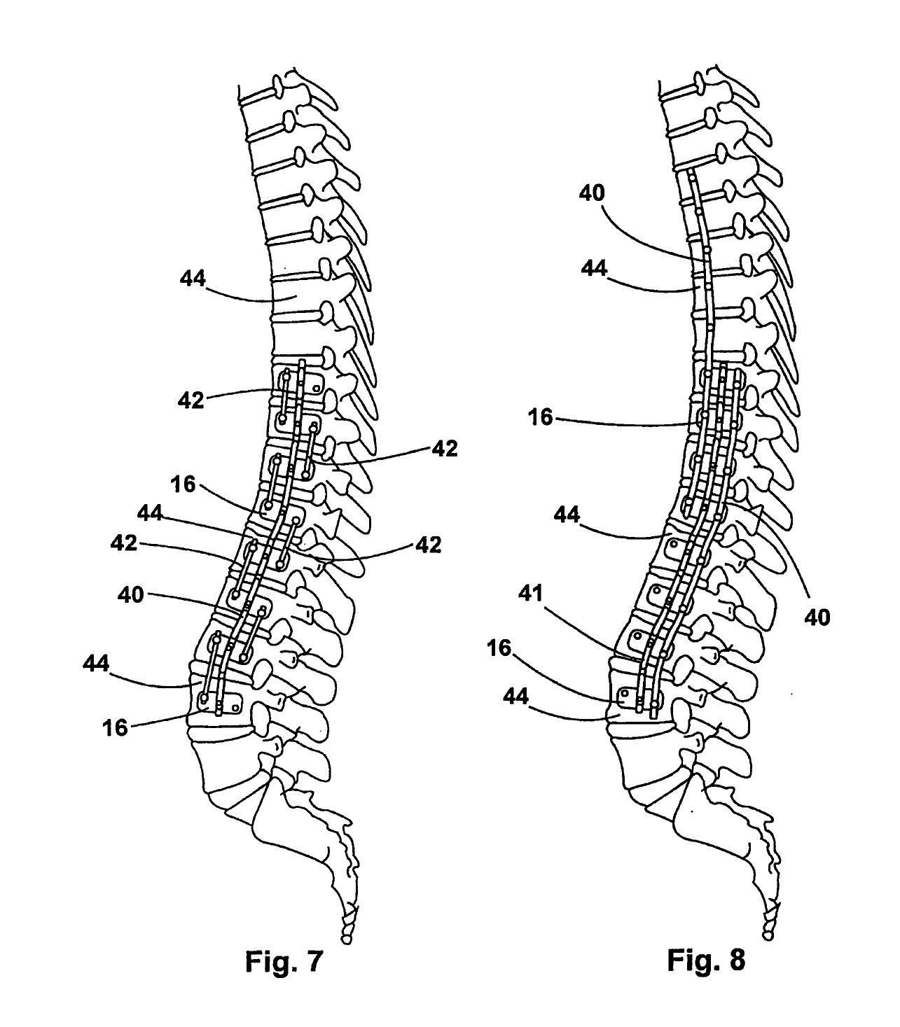

The FIGS. 6, 7 and 8 illustrate various embodiments of the device of the invention in the position in which they are implanted in the spine, all of said embodiments having the same fastening element 16 but being equipped in different ways with fixation rods 12, 40, 42. The fastening element 16 is thereby attached to a ventral vertebral body 44 by means of one or two screws. Depending on the medical situation, the fixation rod 12, 40 may be configured to be a rigid but deformable distraction rod or a compression rod. It is understood that the fixation rod mount 26 of the fastening element 16 and the fixation rod mount 28 of the pedicle screw 14 are configured to match the respective one of the fixation rods 12, 40, 42.

It is also understood that it is not necessary that a fastening element 16 be attached to each vertebral body 44. Depending on the situation, pedicle screws connected with a fixation rod may also be sunk directly into the vertebral body without using a corresponding fastening element or one may skip one vertebral body.

### Listing of Numerals:

10 threaded rod

12 fixation rod

14 pedicle screw

16 fastening element

18 grub screw

20 screw

22 cancellous screw

23 underside

24 spike

25 upper side

26 fixation rod mount

28 threaded rod mount

29, 29′ long hole

30 screw receiving chamber

31 abutment surface

32 screw receiving chamber

33 screw receiving chamber

34 plane

36 plane

40 fixation rod

41 rigid fixation rod

42 fixation rod

44 vertebral body

I claim:

1. A device for aligning a human or animal spine, said device having a fixation rod (12, 40, 42) for fixing at least two vertebral bodies (44), said fixation rod being attachable to at least two fastening elements (16) adapted to be anchored on the spine and being fastenable to the fastening elements (16) by means of screws (20) or other fastening means, characterized in that long holes (29, 29′) through which the screw (20) or the other fastening means for fastening the fixation rod (12, 40, 42) to the fastening element (16) extends are formed in the fixation rod (12, 40, 42) in the axial direction thereof.

2. The device as set forth in claim 1,
characterized in that the spacing between neighboring long holes (29, 29′) is smaller than the length of the shortest of said long holes (29, 29′).

3. The device as set forth in claim 1,
characterized in that two neighboring long holes (29, 29′) have the same length.

4. The device as set forth in claim 1,
characterized in that the long hole (29, 29′) is chamfered to conform to the shape of the screw (20) or of the other fastening means.

5. The device as set forth in claim 1,
characterized in that the fastening element (16) comprises, on its side turned away from the spine, a planar contact surface (31) against which the fixation rod (12, 40, 42) may come to rest.
